(19) United States
(12) Patent Application Publication
(10) Pub. No.: US 2011/0092600 A1
(43) Pub. Date: Apr. 21, 2011

(54) PHARMACEUTICAL COMPOSITIONS COMPRISING AMINOCYCLOHEXANE DERIVATIVES

(75) Inventors: Kirsten Plitt, Mannheim (DE); Brigitte Purmann, Muhlheim (DE); Alda Szlak-Freier, Frankfurt/Main (DE); Bernhard Hauptmeier, Gelnhausen (DE)

(73) Assignee: MERZ PHARMA GMBH & KGAA, FRANKFURT AM MAIN (DE)

(21) Appl. No.: 12/737,283

(22) PCT Filed: Jun. 25, 2009
(86) PCT No.: PCT/EP2009/004599

§ 371 Date: Dec. 23, 2010

Related U.S. Application Data
(60) Provisional application No. 61/133,162, filed on Jun. 26, 2008.

(30) Foreign Application Priority Data
Jun. 26, 2008 (EP) 08011633.8

(51) Int. Cl. A61K 31/13; A61P 25/00; A61P 25/28; A61P 25/16; A61P 25/36; A61P 25/30; A61P 25/32; A61P 25/08; A61P 25/24; A61P 31/12; A61P 31/14

(52) U.S. Cl. 514/579

(57) ABSTRACT

The present invention relates to pharmaceutical compositions comprising 1-aminocyclohexane derivatives and a cyclodextrin, which compositions exhibit advantageous safety, convenience, and dosing characteristics. The compositions of the instant invention find particular application in the treatment of various diseases and conditions of the CNS, including those involving the impairment of cognitive function or dementia, such as Alzheimer's disease.

### FIELD OF THE INVENTION

The present invention relates to pharmaceutical compositions comprising 1-aminocyclohexane derivatives and a cyclodextrin, which compositions exhibit advantageous safety, convenience, and dosing characteristics. The compositions of the instant invention find particular application in the treatment of various diseases and conditions of the CNS, including those involving the impairment of cognitive function or dementia, such as Alzheimer's disease.

### BACKGROUND OF THE INVENTION

Dementia is commonly defined as a chronic deterioration of intellectual function and other cognitive skills severe enough to interfere with the ability to perform activities of daily living. Alzheimer's disease is a form of dementia which is characterized by “ . . . progressive, inexorable loss of cognitive function associated with an excessive number of senile plaques in the cerebral cortex and subcortical gray matter, which also contains β-amyloid and neurofibrillary tangles consisting of tau protein” (Merck Manual 2004). Alzheimer's disease is about twice as common in women as in men, and it accounts for >65% of dementia cases in the elderly. Vascular dementia and Alzheimer's disease coexist in about 15° A) of cases.

Alzheimer's disease is believed to represent the fourth most common medical cause of death. Prevalence of the disease doubles every 5 years beyond age 65 (National Institute on Aging: Prevalence and costs of Alzheimer's disease. Progress Report on Alzheimer's Disease. NIH Publication No. 99 3616, November 1998; Polyikoski et al., Neurology, 2001, 56:1690-1696). Alzheimer's disease currently affects about 15 million people world-wide (including all races and ethnic groups) and, due to the relative increase of elderly people in the population, its prevalence is likely to increase over the next two to three decades.

At present, Alzheimer's disease cannot be cured, i.e. there is no treatment available which effectively reverses its symptoms and progression. Some drugs may, however, alleviate certain symptoms associated with the disease and reduce dependency on care. Among the first drugs approved for the treatment of Alzheimer's disease are members of the class of cholinesterase inhibitors, such as donepezil, galantamine, and tacrine.

1-Aminocyclohexanes, such as neramexane and pharmaceutically acceptable salts thereof, have been found to be useful in the therapy of various diseases especially in certain neurological diseases, including Alzheimer's disease. 1-Aminocyclohexanes, such as neramexane (1-amino-1,3,3,5,5-pentamethylcyclohexane) and pharmaceutically acceptable salts thereof, are disclosed in, e.g., U.S. Pat. Nos. 6,034,134 and 6,071,966 and have been characterized as a low to moderate-affinity, non-competitive NMDA-receptor antagonist believed to selectively block the excitotoxic effects associated with abnormal transmission of glutamate, which is a neurotransmitter that performs an integral role in the neural pathways associated with learning and memory, and which is believed to play a role in Alzheimer's disease.

Oral tablets or capsules represent the most common type of dosage form for oral administration. Typically they are swallowed with liquid such as water. Depending on the specific formulation design, they may also be chewed or dispersed in liquid before administration. Tablets and capsules are considered to be particularly convenient and cost-effective.

Semi-solid and liquid preparations for oral use offer the specific advantages of flexible dosing and easy swallowing. Both aspects have significance in Alzheimer therapy. Swallowing of tablets is difficult for many elderly Alzheimer's patients. Flexible dosing may be recommended in the initial phase of Alzheimer's disease therapy, and, in order to achieve dose reduction for tablet administration, the tablet must be divided in halves, quarters or even more subparts, which may be difficult for some patients.

Liquid formulations may also have certain disadvantages. Since liquid formulations comprise a drug substance in an already dissolved form, the taste and the smell of the compound will be more readily noticed than in the case of solid dosage forms like e.g. a film-coated tablet. If the taste of a drug substance is particularly poor, e.g., if it is bitter or has an astringent or anesthetic effect on oral mucosa, such undesirable properties are usually experienced by a patient in a more pronounced manner if the drug is formulated as a liquid formulation than if the drug is formulated as a film coated tablet or capsule. Moreover, while encapsulation is a common taste-masking technique in solid dosage forms, it is technically very difficult to encapsulate drug substance in a liquid formulation, particularly in the case of compounds having substantial water-solubility. Similarly, a compound having an unpleasant smell may also be difficult to formulate into a palatable liquid preparation. Depending on the intensity of the taste and/or smell of the compound, it may not be sufficient to rely on the incorporation of sweeteners and flavouring agents in order to develop an acceptable aqueous or semi-solid formulation.

Similarly, solid formulations which are not meant to be swallowed as such, but dispersed in liquid such as water or saliva cannot easily be formulated with active compounds having a poor taste or smell.

Conventional aqueous liquid formulations may have other disadvantages, including their potential to enhance the chemical degradation of the drug substance, e.g. by hydrolysis, and thus to lead to a decreased shelf life of the drug product. Moreover, the microbiological stability of an aqueous liquid formulation is typically inferior to that of a solid dosage form such as a tablet or capsule, and, therefore it is often necessary to incorporate preservatives into aqueous liquid formulations.

1-aminocyclohexanes, such as neramexane and pharmaceutically acceptable salts thereof, exhibit taste and smell properties which are problematic and limit the degree of acceptability of conventional liquid or semi-solid formulations, and some patients who might otherwise benefit from liquid formulations may prefer to take these drugs in tablet form.

Thus, a need exists for liquid and/or semi-solid formulations comprising 1-aminocyclohexane derivatives, such as neramexane and pharmaceutically acceptable salts thereof, with improved organoleptic properties like smell and taste.

The instant inventors have discovered that compositions comprising a 1-aminocyclohexane derivative (including neramexane and pharmaceutically acceptable salts thereof) and one or more pharmaceutically acceptable cyclodextrins do not exhibit the disadvantages typically associated with aqueous liquid dosage forms and that such compositions are palatable and acceptable a larger number of patients due to their improved organoleptic properties.

### OBJECTS OF THE INVENTION

It is an object of the invention to provide compositions comprising a 1-aminocyclohexane derivative, such as neramexane and pharmaceutically acceptable salts thereof, and a pharmaceutically acceptable cyclodextrin or a combination of pharmaceutically acceptable cyclodextrins, which compositions exhibit a neutral or sweet taste and little or no unpleasant smell and are remarkably palatable. An additional object of the invention is to provide a water-soluble complex of an 1-aminocyclohexane derivative, such as neramexane and pharmaceutically acceptable salts thereof, and a pharmaceutically acceptable cyclodextrin or cyclodextrin derivative, which complex may be incorporated into a solid, semi-solid or particularly liquid formulations for oral administration which are chemically and microbiologically stable and have a remarkable degree of palatability. It is a further object of the invention to employ such compositions in the treatment of CNS disorders, including Alzheimer's disease. Yet additional objects will become apparent hereinafter, and still further objects will be apparent to one skilled in the art.

### SUMMARY OF THE INVENTION

The present invention relates to a composition comprising a 1-aminocyclohexane derivative selected from those of formula (I)

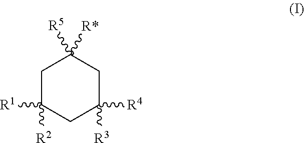

wherein R* is —(CH₂)ₙ—(CR⁶R⁷)ₘ—NR⁸R⁹
wherein n+m=0, 1, or 2
wherein R¹ through R⁷ are independently selected from the group consisting of hydrogen and C₁₋₆alkyl, wherein R⁸ and R⁹ are independently selected from the group consisting of hydrogen and C₁₋₆alkyl or together represent lower-alkylene —(CH₂)ₓ— wherein x is 2 to 5, inclusive, and optical isomers, enantiomers, hydrates, solvates, polymorphs, and pharmaceutically-acceptable salts thereof;
   and a pharmaceutically acceptable cyclodextrin or combination of pharmaceutically acceptable cyclodextrins.

A further aspect of the invention relates to such a composition wherein the pharmaceutically acceptable cyclodextrin is selected from pharmaceutically acceptable, water-soluble, native or derivatised cyclodextrins, such as alpha-cyclodextrin, beta-cyclodextrin, randomly methylated beta-cyclodextrin, 2-O-methyl-beta-cyclodextrin, heptakis-(2,6-di-O-methyl)-beta-cyclodextrin (dimethyl-beta-cyclodextrin), acetylated dimethyl-beta-cyclodextrin, heptakis-(2,3,6-tri-O-methyl)-beta-cyclodextrin (trimethyl-beta-cyclodextrin, sulfoalkylether-beta-cyclodextrin, sulfobutylether-beta-cyclodextrin, O-carboxymethyl-O-ethyl-beta-cyclodextrin, glucuronyl-glucosyl-beta-cyclodextrin, glucosyl-beta-cyclodextrin, maltosyl-beta-cyclodextrin, beta-cyclodextrin sulphate, beta-cyclodextrin phosphate, gamma-cyclodextrin, sulfoalkylether-beta-cyclodextrin, and sulfobutylether-beta-cyclodextrin, and hydroxyalkyl-modified cyclodextrins (including hydroxypropyl-beta-cyclodextrin, e.g., 2-hydroxypropyl-beta-cyclodextrin, or hydroxypropyl-gamma-cyclodextrin, e.g., 2-hydroxypropyl-gamma-cyclodextrin).

A further aspect of the invention relates to such a composition wherein the molar ratio of the cyclodextrin to the 1-aminocyclohexane derivative is at least about 0.1:1.

A further aspect of the invention relates to such a composition wherein the molar ratio of the cyclodextrin to the 1-aminocyclohexane derivative is a maximum of 50:1, (including compositions wherein the molar ratio is 10:1, 5:1, 4:1, 3:1, 2:1, or 1:1) and is at least about 0.1:1.

A further aspect of the invention relates to such a composition wherein the molar ratio of the cyclodextrin to the 1-aminocyclohexane derivative is 3:1.

A further aspect of the invention relates to such a composition in the form of an aqueous liquid composition, such a composition being optionally useful for topical and/or intravitreal application to the eye.

A further aspect of the invention relates to such a composition in the form of a solid composition.

A further aspect of the invention relates to such a composition in the form of an orally disintegrating dosage form or a formulation for reconstitution optionally in form of a powder, granules or a lyophilisate (including such a composition wherein reconstitution, such as the resconstitution of the powder, granules or lyophilisate, with an aqueous solvent results in an aqueous liquid composition).

A further aspect of the invention relates to such a composition, wherein the composition is a rapidly or very rapidly dissolving solid composition, e.g. a powder, a granule or a lyophilisate.

A further aspect of the inventions relates to such a composition, wherein the composition is a rapidly or very rapidly dissolving water soluble powder, a granule or a lyophilisate.

A further aspect of the invention relates to such a composition in the form of a semi-solid composition (including a gel, a cream or an ointment with an acceptable viscosity), such a composition being optionally useful for topical application to the eye.

A further aspect of the invention relates to such a composition wherein the compound of formula (I) is neramexane or a pharmaceutically acceptable salt thereof.

A further aspect of the invention relates to such a composition wherein the concentration of the compound of formula (I) is in the range from about 2 mg/ml to about 100 mg/ml.

A further aspect of the invention relates to such a composition wherein the composition further comprises at least one flavouring agent or taste-masking agent other than a cyclodextrin.

A further aspect of the invention relates to such a composition wherein the composition is substantially free of preservatives.

A further aspect of the invention relates to such a composition wherein the composition further comprises a preservative and the concentration of the preservative is below the concentration needed to effectively preserve an equivalent placebo composition.

A further aspect of the invention relates to such a composition wherein the composition further comprises at least one additional active ingredient.

A further aspect of the invention relates to such a composition, wherein the composition further comprises at least one additional active ingredient selected from acetylcholinesterase inhibitors.

An further aspect of the invention relates to such a composition wherein the pharmaceutically acceptable cyclodextrin is selected from pharmaceutically acceptable, water-soluble, native or derivatised cyclodextrins, such as such as alpha-cyclodextrin, beta-cyclodextrin, randomly methylated beta-cyclodextrin, 2-O-methyl-beta-cyclodextrin, heptakis-(2,6-di-O-methyl)-beta-cyclodextrin (dimethyl-beta-cyclodextrin), acetylated dimethyl-beta-cyclodextrin, heptakis-(2,3,6-tri-O-methyl)-beta-cyclodextrin (trimethyl-beta-cyclodextrin, sulfoalkylether-beta-cyclodextrin, sulfobutylether-beta-cyclodextrin, O-carboxymethyl-O-ethyl-beta-cyclodextrin, glucuronyl-glucosyl-beta-cyclodextrin, glucosyl-beta-cyclodextrin, maltosyl-beta-cyclodextrin, beta-cyclodextrin sulphate, beta-cyclodextrin phosphate, gamma-cyclodextrin, sulfoalkylether-beta-cyclodextrin, and sulfobutylether-beta-cyclodextrin, and hydroxyalkyl-modified cyclodextrins (including hydroxypropyl-beta-cyclodextrin, e.g., 2-hydroxypropyl-beta-cyclodextrin, or hydroxypropyl-gamma-cyclodextrin, e.g., 2-hydroxypropyl-gamma-cyclodextrin).

A further aspect of the invention relates to a pharmaceutical composition comprising any of the above-described compositions in combination with one or more additional pharmaceutically acceptable excipients.

An additional aspect of the invention relates to such a pharmaceutical composition in the form of solid composition.

An additional aspect of the invention relates to such a pharmaceutical composition in the form of an aqueous liquid composition.

An additional aspect of the invention relates to such a pharmaceutical composition in the form of a semi-solid composition.

A further aspect of the invention relates to a medicament comprising any of the above-described compositions.

A further aspect of the invention relates to the use of the above-described compositions in treating CNS as well as other disorders, including hypoxia, hypoglycemia, hepatic encephalopathy, chronic neurodegenerative diseases, dementia, Alzheimer's disease, vascular dementia, Parkinson's disease, Huntington's disease, multiple sclerosis, amyotrophic lateral sclerosis, AIDS-neurodegeneration, AIDS-related dementia, olivopontocerebellar atrophy, Tourette's syndrome, motor neurone disease, mitochondrial dysfunction, Korsakoff syndrome, Creutzfeldt-Jakob disease, chronic pain, acute pain, drug tolerance, dependence and addiction (e.g., opioids, cocaine, benzodiazepines, and alcohol), neuropathic pain, epilepsy, depression, anxiety, schizophrenia, spasticity, nystagmus, ocular diseases, tinnitus, hepatic encephalopathy, multiple sclerosis, stroke, dyskinesia, malaria, and viral infections such as hepatitis C and Borna virus, conditions requiring an immunomodulator, emesis, drug and alcohol abuse disorders, cognitive disorders, cerebellar tremor, and appetite disorders.

A further aspect of the invention relates to a composition as defined above for the treatment of CNS disorders as well as other disorders including hypoxia, hypoglycemia, hepatic encephalopathy, chronic neurodegenerative diseases, dementia, Alzheimer's disease, vascular dementia, Parkinson's disease, Huntington's disease, multiple sclerosis, amyotrophic lateral sclerosis, AIDS-neurodegeneration, AIDS-related dementia, olivopontocerebellar atrophy, Tourette's syndrome, motor neurone disease, mitochondrial dysfunction, Korsakoff syndrome, Creutzfeldt-Jakob disease, chronic pain, acute pain, drug tolerance, dependence and addiction (e.g., opioids, cocaine, benzodiazepines, and alcohol), neuropathic pain, epilepsy, depression, anxiety, schizophrenia, spasticity, nystagmus, ocular diseases, tinnitus, hepatic encephalopathy, multiple sclerosis, stroke, dyskinesia, malaria, and viral infections such as hepatitis C and Borna virus, conditions requiring an immunomodulator, emesis, drug and alcohol abuse disorders, cognitive disorders, cerebellar tremor, and appetite disorders.

A further aspect of the invention relates to a method of treating CNS as well as other disorders, including hypoxia, hypoglycemia, hepatic encephalopathy, chronic neurodegenerative diseases, dementia, Alzheimer's disease, vascular dementia, Parkinson's disease, Huntington's disease, multiple sclerosis, amyotrophic lateral sclerosis, AIDS-neurodegeneration, AIDS-related dementia, olivopontocerebellar atrophy, Tourette's syndrome, motor neurone disease, mitochondrial dysfunction, Korsakoff syndrome, Creutzfeldt-Jakob disease, chronic pain, acute pain, drug tolerance, dependence and addiction (e.g., opioids, cocaine, benzodiazepines, and alcohol), neuropathic pain, epilepsy, depression, anxiety, schizophrenia, spasticity, nystagmus, ocular diseases, tinnitus, hepatic encephalopathy, multiple sclerosis, stroke, dyskinesia, malaria, and viral infections such as hepatitis C and Borna virus, conditions requiring an immunomodulator, emesis, drug and alcohol abuse disorders, cognitive disorders, cerebellar tremor, and appetite disorders, in a subject in need thereof, comprising administering an effective amount of a composition as described above.

Non-limiting examples of the 1-aminocyclohexane derivatives of formula (I) used according to the present invention include:
1-amino-1,3,5-trimethylcyclohexane,
1-amino-1(trans),3(trans),5-trimethylcyclohexane,
1-amino-1(cis),3(cis),5-trimethylcyclohexane,
1-amino-1,3,3,5-tetramethylcyclohexane,
1-amino-1,3,3,5,5-pentamethylcyclohexane (neramexane),
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-1,3,3,5,5-pentamethylcyclohexane,
N-ethyl-1-amino-1,3,3,5,5-pentamethyl-cyclohexane,
N-(1,3,3,5,5-pentamethylcyclohexyl)pyrrolidine,
3,3,5,5-tetramethylcyclohexylmethylamine,
1-amino-1-propyl-3,3,5,5-tetramethylcyclohexane,
1 amino-1,3,3,5(trans)-tetramethylcyclohexane (axial amino group),
3-propyl-1,3,5,5-tetramethylcyclohexylamine semihydrate,
1-amino-1,3,5,5-tetramethyl-3-ethylcyclohexane,
1-amino-1,3,5-trimethylcyclohexane,
1-amino-1,3-dimethyl-3-propylcyclohexane,
1-amino-1,3(trans),5(trans)-trimethyl-3(cis)-propylcyclohexane,
1-amino-1,3-dimethyl-3-ethylcyclohexane,
1-amino-1,3,3-trimethylcyclohexane,
cis-3-ethyl-1(trans)-3(trans)-5-trimethylcyclohexamine,
1-amino-1,3(trans)-dimethylcyclohexane,
1,3,3-trimethyl-5,5-dipropylcyclohexylamine,
1-amino-1-methyl-3(trans)-propylcyclohexane,
1-methyl-3(cis)-propylcyclohexylamine,
1-amino-1-methyl-3(trans)-ethylcyclohexane,
1-amino-1,3,3-trimethyl-5(cis)-ethylcyclohexane,
1-amino-1,3,3-trimethyl-5(trans)-ethylcyclohexane,
cis-3-propyl-1,5,5-trimethylcyclohexylamine,
trans-3-propyl-1,5,5-trimethylcyclohexylamine,
N-ethyl-1,3,3,5,5-pentamethylcyclohexylamine,
N-methyl-1-amino-1,3,3,5,5-pentamethylcyclohexane,
1-amino-1-methylcyclohexane,
N,N-dimethyl-1-amino-1,3,3,5,5-pentamethylcyclohexane,
2-(3,3,5,5-tetramethylcyclohexyl)ethylamine,
2-methyl-1-(3,3,5,5-tetramethylcyclohexyl)propyl-2-amine,
2-(1,3,3,5,5-pentamethylcyclohexyl)-ethylamine semihydrate,
N-(1,3,3,5,5-pentamethylcyclohexyl)-pyrrolidine,
1-amino-1,3(trans),5(trans)-trimethylcyclohexane,
1-amino-1,3(cis),5(cis)-trimethylcyclohexane,
1-amino-(1R,5S)trans-5-ethyl-1,3,3-trimethylcyclohexane,
1-amino-(1S,5S)cis-5-ethyl-1,3,3-trimethylcyclohexane,
1-amino-1,5,5-trimethyl-3(cis)-isopropyl-cyclohexane,
1-amino-1,5,5-trimethyl-3(trans)-isopropyl-cyclohexane,
1-amino-1-methyl-3(cis)-ethyl-cyclohexane,
1-amino-1-methyl-3(cis)-methyl-cyclohexane,
1-amino-5,5-diethyl-1,3,3-trimethyl-cyclohexane,
1-amino-1,3,3,5,5-pentamethylcyclohexane,
1-amino-1,5,5-trimethyl-3,3-diethylcyclohexane,
1-amino-1-ethyl-3,3,5,5-tetramethylcyclohexane,
N-ethyl-1-amino-1,3,3,5,5-pentamethylcyclohexane,
N-(1,3,5-trimethylcyclohexyl)pyrrolidine or piperidine,
N-[1,3(trans),5(trans)-trimethylcyclohexyl]pyrrolidine or piperidine,
N-[1,3(cis),5(cis)-trimethylcyclohexyl]pyrrolidine or piperidine,
N-(1,3,3,5-tetramethylcyclohexyl)pyrrolidine or piperidine,
N-(1,3,3,5,5-pentamethylcyclohexyl)pyrrolidine or piperidine,
N-(1,3,5,5-tetramethyl-3-ethylcyclohexyl)pyrrolidine or piperidine,
N-(1,5,5-trimethyl-3,3-diethylcyclohexyl)pyrrolidine or piperidine,
N-(1,3,3-trimethyl-cis-5-ethylcyclohexyl)pyrrolidine or piperidine,
N-[(1S,5S)cis-5-ethyl-1,3,3-trimethylcyclohexyl]pyrrolidine or piperidine,
N-(1,3,3-trimethyl-trans-5-ethylcyclohexyl)pyrrolidine or piperidine,
N-[(1R,5S)trans-5-ethyl,3,3-trimethylcyclohexyl]pyrrolidine or piperidine,
N-(1-ethyl-3,3,5,5-tetramethylcyclohexyl)pyrrolidine or piperidine,
N-(1-propyl-3,3,5,5-tetramethylcyclohexyl)pyrrolidine or piperidine,
N-(1,3,3,5,5-pentamethylcyclohexyl)pyrrolidine,
   and optical isomers, diastereomers, enantiomers, hydrates, their pharmaceutically acceptable salts, and mixtures thereof.

### DETAILED DESCRIPTION OF THE INVENTION

As used herein, the term aqueous liquid composition means a liquid preparation wherein the major liquid component is water. Optionally, the aqueous liquid composition may further comprise other liquid components, such as pharmaceutically acceptable organic solvents and cosolvents. Examples of such other liquid components are e.g. ethanol, glycerol, propylene glycol, and polyethylene glycol. Such water-miscible organic solvents and cosolvents may be incorporated for example in order to solubilise a poorly water-soluble ingredient, such as a lipophilic substance.

The term liquid formulation includes liquid solutions and dispersions, such as emulsions and suspensions.

As used herein, the term semi-solid composition means a preparation with low viscosity whose major liquid component is water. Optionally, the semi-solid composition may further comprise other liquid components, such as pharmaceutically acceptable organic solvents, cosolvents, viscosity regulation polymers and emulsifiers. Examples of such other liquid components are ethanol, glycerol, propylene glycol, and polyethylene glycol. Such water-miscible organic solvents may be incorporated for example in order to solubilise a poorly water-soluble ingredient, such as a lipophilic substance.

The term semi-solid composition includes gels, creams and ointments. In comparison to a liquid composition these formulations are not freely flowing. The viscosity of semi-solid compositions may be controlled by one polymer or by a combination of polymers like acacia gum and derivatives, alginic acid and derivatives, carbomer and derivatives, carboxymethylcellulose and derivatives, carrageenan and derivatives, croscarmellose and derivatives, crospovidone and derivatives, dextrin and derivatives, ethylcellulose and derivatives, gelatin and derivatives, guar gum and derivatives, hydroxyethyl cellulose and derivatives, hypromellose and derivatives, hydroxypropyl methylcellulose and derivatives, lecithin and derivatives, maltodextrin and derivatives, methylcellulose and derivatives, poloxamer and derivatives, polethylenglycoles and derivatives, polymethacrylates and derivatives, polyoxyethylalkylethers and derivatives, polyvinylalkohol, polyvinylpyrrolidon and derivatives, silicon dioxide and derivatives, sodium starch glycolate and derivatives, sorbitol and derivatives, starch and derivatives, pregelatinised starch and derivatives, tragacanth and derivatives and xanthan gum and derivatives,

As used herein, the term solid composition includes hard capsules, soft capsules, tablets, coated tablets, lozenges, wafers, granules, powders, lyophilisates and the like.

1-Aminocyclohexane derivatives of formula (I) (e.g., neramexane, 1-amino-1,3,3,5,5-pentamethylcyclohexane) are disclosed in U.S. Pat. Nos. 6,034,134 and 6,071,966. Compounds of formula (I) (e.g., neramexane) may be used according to the invention in the form of any of pharmaceutically acceptable salts, solvates, isomers, conjugates, and prodrugs, any references to compounds of formula (I) (e.g., neramexane) in this description should be understood as also referring to such salts, solvates, isomers, conjugates, and prodrugs.

The term “analog” or “derivative” is used herein in the conventional pharmaceutical sense, to refer to a molecule that structurally resembles a reference molecule (such as neramexane), but has been modified in a targeted and controlled manner to replace one or more specific substituents of the referent molecule with an alternate substituent, thereby generating a molecule which is structurally similar to the reference molecule. Synthesis and screening of analogs (e.g., using structural and/or biochemical analysis), to identify slightly modified versions of a known compound which may have improved or biased traits (such as higher potency and/or selectivity at a specific targeted receptor type, greater ability to penetrate mammalian blood-brain barriers, fewer side effects, etc.) is a drug design approach that is well known in pharmaceutical chemistry.

Pharmaceutically acceptable salts include, but are not limited to, acid addition salts, such as those made with hydrochloric, methylsulfonic, hydrobromic, hydroiodic, perchloric, sulfuric, nitric, phosphoric, acetic, propionic, glycolic, lactic, pyruvic, malonic, succinic, fumaric, tartaric, citric, benzoic, carbonic, cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid. All of these salts (or other similar salts) may be prepared by conventional means. The nature of the salt is not critical, provided that it is non-toxic and does not substantially interfere with the desired pharmacological activity.

Cyclodextrins are oligosaccharides composed of glucopyranose units. The major unsubstituted or native cyclodextrins are usually prepared by the enzymatic degradation of starch. They are cone-like, toroidal molecules with a rigid structure and a central cavity, the size of which varies according to the cyclodextrin type. The internal cavity of cyclodextrins is more hydrophobic and capable of accommodating both lipophilic and hydrophilic molecules in the form of inclusion complexes, thus enabling, for example, the solubilisation of poorly soluble drugs in aqueous media. Other types of complexes involving cyclodextrins have recently been identified.

As used herein, a complex means an association of molecules by non-covalent interactions. Complexation taking place in solution is typically an equilibrium process. However, complexes may also occur in solid state. An inclusion complex is a structure wherein a guest molecule is either partially or completely contained within a cavity of a larger host molecule.

The three major types of pharmaceutically acceptable cyclodextrins are alpha-, beta- and gamma-cyclodextrins, comprising 6, 7 and 8 glucopyranose units, respectively. In addition, a number of chemically modified cyclodextrins have been developed, mostly motivated by the desire to increase the water solubility and extend their usefulness as solubilising excipients. Examples of such derivatives include alpha-cyclodextrin, beta-cyclodextrin, randomly methylated beta-cyclodextrin, 2-O-methyl-beta-cyclodextrin, heptakis-(2,6-di-O-methyl)beta-cyclodextrin (dimethyl-beta-cyclodextrin), acetylated dimethyl-beta-cyclodextrin, heptakis-(2,3,6-tri-O-methyl)-beta-cyclodextrin (trimethyl-beta-cyclodextrin, sulfoalkylether-beta-cyclodextrin, sulfobutylether-beta-cyclodextrin, O-carboxymethyl-O-ethyl-beta-cyclodextrin, glucuronyl-glucosyl-beta-cyclodextrin, glucosyl-beta-cyclodextrin, maltosyl-beta-cyclodextrin, beta-cyclodextrin sulphate, beta-cyclodextrin phosphate, gamma-cyclodextrin, sulfoalkylether-beta-cyclodextrin, and sulfobutylether-beta-cyclodextrin (SBEBCD), and hydroxyalkyl-modified cyclodextrins (including hydroxypropyl-beta-cyclodextrin, e.g., 2-hydroxypropyl-beta-cyclodextrin (HPBCD), or hydroxypropyl-gamma-cyclodextrin, e.g., 2-hydroxypropyl-gamma-cyclodextrin (HPGCD)). As used herein, the term “cyclodextrins” includes such modified versions of the native cyclodextrins.

An example of a suitable grade of hydroxypropyl-beta-cyclodextrin is amorphous, randomly substituted hydroxypropyl-beta-cyclodextrin with a degree of substitution DS in the range of about 4.5 (i.e. between approx. 4 and 5), such as the product marketed as Kleptose™ HPB (Roquette). It is noted that the DS value, as used herein, defines the average number of substituted hydroxyl groups per anhydroglucose unit, not per cyclodextrin molecule. Other examples of useful grades are randomly substituted hydroxypropyl-beta-cyclodextrin with a degree of substitution DS in the range of about 5.6, or in the range of 2 to 4, or in the range of 5, or in the range of 6.5, respectively. An example of a suitable grade of hydroxypropyl-gamma-cyclodextrin is the product marketed as Cavasol™ W8 HP (Wacker Chemie).

The amount of cyclodextrin in the composition may be selected taking into account the type of cyclodextrin and the concentration of the active ingredient which is to be achieved. The concentration of the active ingredient may be at least about 1 mg/ml. If the active ingredient is a neramexane compound, such as neramexane base, neramexane hydrochloride or neramexane mesylate, the concentration may be in the range from about 2 to about 100 mg/ml. Alternatively, the pharmaceutical compositions may comprise neramexane, or a pharmaceutically acceptable salt thereof, in a range of 2 to 50 mg/ml. For example, a concentration of 5-10 mg/ml of neramexane, or a pharmaceutically acceptable salt thereof, may be formulated in such a composition.

According to the present invention, palatable compositions (for example liquid, semi solid or solid compositions) may be obtained when the amount of the cyclodextrin is selected to give a molar cyclodextrin-to-active ratio of at least about 0.5:1 to a maximum of 100:1. Another embodiment may comprise a cyclodextrin-to-active ratio of at least about 1:1. Other embodiments may comprise a ratio of about 0.75:1 to 50:1 (including 5:1, 10:1, and 25:1). The molar ratio of the cyclodextrin to the drug substance may also be formulated from about 1:1 to about 10:1, such as from about 2:1 to about 10:1, such as about 2:1, 3:1, or about 6:1, or about 7:1, or about 8:1, or about 9:1, respectively.

It has been found that such ratios lead to a remarkably high degree of taste masking, which may be associated with the spontaneous formation of a soluble complex between the cyclodextrin molecule and the drug substance. Moreover, the unpleasant smell of the active compound, in particular in the case of a neramexane compound, is substantially reduced. The degree of taste-masking is surprising in view of the high water-solubility of the active compounds, particularly in the case of readily soluble salt forms, such as neramexane mesylate. It is also surprising that the complexation achieves effective taste-masking without eliminating other properties of the drug substance which are believed to result from their state of being dissolved in the aqueous solvent. After oral administration, for example, the active compound is rapidly absorbed from the composition and becomes bioavailable without any delay.

If the molar ratio of the cyclodextrin to the active compound is selected to provide substantial but not complete taste masking, which may occur if the ratio is chosen in the lower part of the ranges given above (e.g., a ratio of 1:1), the addition of one or more further excipients which improve the palatability of the formulation may also be contemplated. This is particularly true for neramexane mesylate solutions in the preferred drug concentration range. For example, one or more sweeteners may be incorporated. Furthermore, one or more excipients selected from the group of flavours, flavour enhancers, and taste masking agents may be added, with the proviso that such taste-masking agents are not selected from the class of cyclodextrins.

Sweeteners, as used herein, are natural or synthetic compounds which have a sweet taste and are physiologically acceptable. Examples of natural sweeteners include common sugars and sugar alcohols such as sucrose, glucose, fructose, maltose, maltitol, xylitol, lactitol, mannitol, and sorbitol. A sugar alcohol may be used to improve the flavour of the composition of the invention, for example sorbitol. A useful concentration range for sorbitol or other sugars and sugar alcohols is from about 5% (w/v) to about 25% (w/v), a 10% (w/v) may also be used.

In another embodiment, an artificial sweetener is incorporated in the composition in addition to, or instead of, a natural sweetener. Useful artificial sweeteners include saccharin-sodium, saccharin, sodium cyclamate, acesulfame K, neohesperidine dihydrochalcone, and aspartame, as well as any other sweeteners whose safety in human use is established. Appropriate concentrations depend on the individual sweetener which is selected, but also on the specific cyclodextrin which is chosen. For example, hydroxypropyl-beta-cyclodextrin already provides for a rather sweet taste so that the addition of a sweetening agent may not increase the palatability of the formulation any further.

Suitable flavors which may further improve the taste of cyclodextrin-containing aqueous compositions of aminocyclohexane derivatives (including neramexane and pharmaceutically acceptable salts thereof) include grape, orange, peppermint, spearmint, cherry, liquorice, and aniseed. In particular, peppermint flavours are physicochemically and organoleptically well-compatible with the key components of the composition of the invention and may lead to palatable formulations.

If an oily flavouring component (such as peppermint oil) is used, it may be necessary or useful to also add an excipient which can contribute to the solubilisation of the oil in the aqueous phase. For example, a surfactant or a water-miscible organic cosolvent may be used for this purpose. In the case of peppermint oil or other peppermint flavouring products, solubilisation may be achieved by the incorporation of propylene glycol.

For liquid compositions it may be useful that the complex of the invention leads to a strong taste-masking effect while not inhibiting the preservative effect of the active ingredient. As a result of the retained antimicrobial effect of the drug substance, it may be possible to formulate the composition of the invention without any additional preservative. In one of the embodiments, therefore, the composition of the invention is indeed substantially free of preservatives. In this context, the term “substantially” means that preservatives are not detectable in the composition, or only in concentrations which are generally considered irrelevant with regard to any preservation effects.

The liquid composition may, optionally, comprise at least one preservative, but at a concentration which is insufficient to effectively preserve an equivalent placebo composition. As used herein, an equivalent placebo composition is defined as a composition, substantially free of active ingredients, whose properties and other ingredients are largely the same as those of the drug-containing reference composition.

Whether a composition is effectively preserved may be determined according to tests known to those skilled in the art, such as the test for preservative efficacy (USP <51>), wherein five challenge organisms are added to test compositions at defined time intervals, depending on the product category. Conducted in appropriate series, such testing can also be performed in order to determine the minimally effective concentration of a specific preservative for a given composition, such as a drug-free composition equivalent to a composition according to the invention. For example, it may be found that in order to effectively preserve a particular placebo composition with sorbic acid, the preservative must be present at a concentration of at least about 0.1% (w/v). In this case, the reference composition which comprises the 1-aminocyclohexane derivative could contain sorbic acid at a substantially lower concentration, such as about 0.05% (w/v) or less. In another embodiment, the concentration of the preservative is selected to be not more than about a fifth, including not more than about a tenth, of the concentration needed to effectively preserve an equivalent placebo composition.

For reproducible product quality and reliable stability, the composition may be adjusted to a specific pH by incorporating one or more appropriate excipients selected from the group consisting of physiologically acceptable acids, bases, and acidic and alkaline salts. For example, the combination of citric acid and sodium citrate may be used for buffering the pH of the composition at a value selected in the range from about pH 4 to about pH 10. In particular, the pH may be adjusted to a value from about pH 4.5 to about pH 8, using pharmaceutically acceptable buffering agents or mixtures such as citrate buffer.

Further excipients which are routinely used in pharmaceutical formulations may be incorporated as may seem appropriate to adjust the composition to the specific requirements of a particular drug candidate, or to a specific use or target population. Examples of potentially suitable excipients are thickeners such as soluble gums including carrageenan, alginate, xanthan, and soluble cellulose esters; colouring agents; stabilizers, such as antioxidants, or crystallization inhibitors, such as glycerol, propylene glycol, or polyvinylpyrrolidone.

Optionally, the composition may further comprise another active ingredient which is not an aminocyclohexane derivative. As used herein, an active ingredient is a pharmaceutically acceptable compound or mixture of compounds useful for the diagnosis, prevention, or treatment of a symptom, disease, or condition. The terms “active compound”, “active ingredient”, “drug”, and “drug substance” may be used interchangeably. Additional active ingredients include acetylcholinesterase inhibitors, such as donepezil, rivastigmine, tacrine, galantamine, physostigmine, huperzine A, zanapezil, ganstigmine, phenserine, phenethylnorcymserine (PENC), cymserine, thiacymserine, SPH 1371 (galantamine plus), ER 127528, RS 1259, and F3796.

It has been surprisingly found that the complex between neramexane and its pharmaceutically acceptable salts and one cyclodextrin or a combination of cyclodextrins is formed spontaneously in aqueous media. The complex formation takes place with all types of native cyclodextrins and cyclodextrin derivatives, including beta- and gamma-cyclodextrin as well as hydroxypropyl-beta- or hydroxypropyl-gamma-cyclodextrin.

The complex formation takes place spontaneously in aqueous solutions at normal room temperature, which is in contrast to many other known cyclodextrin complexes whose preparation requires the application of heat and/or very long stirring times. Hence, the preparation of the composition is technically easy, quick and cost-efficient. In most cases, the components are simply weighed and combined with a measured amount of water or cosolvents followed by stirring until dissolution occurs. The mixture may be agitated and/or heated. The solution may be further processed by filtration or centrifugation to remove residual particles. If a solid complex is desired, the solution may be dried, such as by spray drying or freeze drying.

The complex formation may also be achieved by conventional single pot wet granulation processes or fluid bed granulation processes using only very limited amounts of aqueous media or organic solvents or cosolvents or mixtures of them. The complex can also be formed by spray drying the neramexane cyclodextrin solution. For the conventional granulation processes neramexane and the cyclodextrin can be blended homogeneously and then be granulated with an aqueous solution or a cosolvent containing aqueous solution. The granulation process can also be performed in that way, that neramexane is granulated with a cyclodextrin solution or that a cyclodextrin powder is granulated with a neramexane solution. Only in the case of adding a water-insoluble ingredient it may be useful to first dissolve that excipient in an amount of surfactant or cosolvent before combining it with the remaining components.

In general, various methods may be used to make such cyclodextrin/drug complex, such as the solution method, the co-precipitation method, the slurry method, the kneading method and the grinding method (T Loftsson, Pharmaceutical Technology 12, 41-50, 1999). The slurry method and the kneading method are more often used for the complexation of poorly water-soluble compounds, whereas the solution and the precipitation method may easily be used for both water-soluble and poorly-soluble compounds.

The present liquid and semi-solid compositions may be filled into a container which holds a plurality of doses. Appropriate containers will hold a volume in the range from about 5 ml or 5 g to about 1,000 ml or 1,000 g, and other containers may hold from about 10 ml (or g) to about 500 ml (or g). The volume is selected in consideration of the strength of the specific formulation and the time period for which the product is to be used. For example, a container may be selected to accommodate the medication needed for several days, weeks, or months. In one of the preferred embodiments, the container is selected to hold sufficient medication for at least about 4 weeks. In another embodiment, the container is selected to hold about 50 ml (or g), about 100 ml (or g), about 200 ml (or g), about 250 ml (or g), or about 500 ml (or g).

Appropriate containers may be of glass or a suitable plastic, such as polypropylene or polyethylene, and will usually have a closure which is reclosable. Optionally, the closure system is child-proof.

The container may further comprise a means for measuring and/or dispensing defined doses of the composition. A conventional measuring means is, for example, a dropper, i.e. a glass tube fitted with a rubber bulb which is integrated in the closure and removed when opening the container. Alternatively, a non-removable dropper may be integrated in the bottle neck.

The container, or container system, may also comprise a dosing cup that provides markings indicating the amount of liquid to be taken for the most common doses. For example, the markings may range from about 0.5 ml to about 10 ml, and from about 1 ml to about 5 ml, or instead of volumes they may indicate the dose in grams of formulation, or in mg of drug substance. The measuring cup may be part of the container closure system, or it may be provided as a separate device within the secondary package in which the container is presented.

The composition of the invention may be a solid composition. Moreover, the 1-aminocyclohexane derivative and the cyclodextrin may be present in the solid composition in already complexed form, or the composition may comprise both components in non-complexed form and the complex forms spontaneously when the composition is used in an aqueous environment.

The composition of the present invention may be in the form of a preparation for reconstitution, such as a powder, a granulate, a tablet, a wafer or a lyophilisate for the preparation of a ready-to-use liquid or semi solid formulation for oral administration. In this case, the composition may be composed in the same manner as described above in the context of a liquid composition in view of the same effects and advantages, except that it does not comprise a liquid. Instead, such product is designed to be combined with a liquid, such as water or a mixture of water and a cosolvent, before administration. In comparison with a ready-to-use liquid composition, such solid composition for reconstitution is potentially more stable and may exhibit a longer shelf-life. It is also lighter and less costly to ship and store; however, a ready-to-use liquid formulation may be more convenient for those patients who prefer not to have to manipulate the formulation before administration.

A solid composition for reconstitution may be prepared by conventional pharmaceutical processing steps. For example, it may be prepared in a similar manner as the liquid composition described above, and subsequently dried, e.g. by lyophilisation or spray drying.

Alternatively, the solid composition may be designed and formulated for oral administration as such, without reconstitution. In this context, oral administration is understood as comprising all forms of oral use, including intraoral and peroral administration. An example of dosage forms for intraoral administration are those formulations which disintegrate in the mouth rather than in the gastric fluid, which formulations are also known as orally disintegrating dosage forms, fast-melt tablets and oral wafers. Examples of formulations for peroral administration include conventional hard or soft capsules and tablets.

As used herein, in conjunction with the compositions of the present invention, the term “rapidly dissolving” means the release of a 1-aminocyclohexane derivative (e.g., neramexane or a pharmaceutically acceptable salt thereof) of at least 85% within 30 minutes, as determined using a paddle (50 rpm) or basket (100 rpm) apparatus at a temperature of about 37° C. and a volume of about 900 ml, and the term “very rapidly dissolving” means the release of a 1-aminocyclohexane derivative (e.g., neramexane or a pharmaceutically acceptable salt thereof) of at least 85% within 15 minutes, as determined using a paddle (50 rpm) or basket (100 rpm) apparatus at a temperature of about 37° C. and a volume of about 900 ml.

As used herein, the term medicament includes a drug product which may represent the composition as such or in combination with a container or other packaging means, a dosing device, a liquid for reconstitution, or with another drug substance-containing composition in the form of a kit or combination package.

According to some embodiments, the present invention relates to administration of compositions comprising a 1-aminocyclohexane derivative (including neramexane and pharmaceutically acceptable salts thereof), to an individual in need thereof. For example, the compositions of the invention are suitable for the treatment of CNS as well as other disorders, including hypoxia, hypoglycemia, hepatic encephalopathy, chronic neurodegenerative diseases, dementia, Alzheimer's disease, vascular dementia, Parkinson's disease, Huntington's disease, multiple sclerosis, amyotrophic lateral sclerosis, AIDS-neurodegeneration, AIDS-related dementia, olivopontocerebellar atrophy, Tourette's syndrome, motor neurone disease, mitochondrial dysfunction, Korsakoff syndrome, Creutzfeldt-Jakob disease, chronic pain, acute pain, drug tolerance, dependence and addiction (e.g., opioids, cocaine, benzodiazepines, and alcohol), neuropathic pain, epilepsy, depression, anxiety, schizophrenia, spasticity, nystagmus, ocular diseases, tinnitus, hepatic encephalopathy, multiple sclerosis, stroke, dyskinesia, malaria, and viral infections such as hepatitis C and Borna virus, conditions requiring an immunomodulator, emesis, drug and alcohol abuse disorders, cognitive disorders, cerebellar tremor, and appetite disorders.

The term “treat” is used herein to mean to relieve or alleviate at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

The term “therapeutically effective” applied to dose or amount refers to that quantity of a compound or pharmaceutical composition that is sufficient to result in a desired activity upon administration to a mammal in need thereof.

The term “nystagmus” as used herein encompasses congenital and acquired forms of the disease, including subtypes thereof. Further the term nystagmus encompasses pathological forms of the disease and nystagmus resulting from toxic or metabolic causes, including subtypes thereof. The term nystagmus also includes ocular tremor or oscillopsia. Moreover, nystagmus also encompasses downbeat nystagmus, upbeat nystagmus, seesaw nystagmus, periodic alternating nystagmus, and acquired pendular nystagmus. Conditions/diseases to be mentioned as falling under the category “congenital nystagmus” include, but are not limited to, idiopathy, albinism, aniridia, Leber's congenital amaurosis, bilateral optic nerve hypoplasia, bilateral congenital cataracts, rod monochromatism, optic nerve or macular disease, persistent tunica vasculosa lentis, latent nystagmus and nystagmus blockage syndrome. Examples of diseases/conditions falling under the definition of “pathological nystagmus” include, but are not limited to, peripheral nystagmus, positional nystagmus, gaze induced nystagmus, post head shake nystagmus, spontaneous nystagmus as well as central nystagmus. Conditions/disorders falling under the definition “acquired nystagmus” include, but are not limited to benign paroxysmal positional vertigo, head trauma, stroke, Ménière's disease and other balance disorders, multiple sclerosis, brain tumors, Wernicke-Korsakoff syndrome, encephalopathy, lateral medullary syndrome, optic nerve hypoplasia, Nooan syndrome, Pelizaeus-Merzbacher disease, superior canal dehiscence syndrome, tullio phenomenon, Horner's syndrome. Conditions/disorders falling under the definition “Nystagmus resulting from toxic or metabolic causes” include, but are not limited to intoxications with alcohol, lithium, barbiturates, phenyloin, salicylates, benzodiazepines, LSD, phenylcyclidine, aminoglycosides, anticonvulsants, sedatives, methylenedioxymethamphetamine, Wernicke's encephalopathy, thiamine deficiency.

The term “ocular diseases” as used herein includes ocular hypertension, glaucoma, low-tension glaucome, diabetic retinopathy, age-related macular degeneration, diabetic macular edema, ischemic optic neuropathy, optic nerve trauma, optic neuritis, retinal vein occlusion, retinal artery occlusion, retinal edema, retinal ischemia, damages of the retina caused by e.g. photocoagulation and accidental laser injuries.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). The term “pharmaceutically acceptable” may also mean approved by a regulatory agency of the Federal or a state government or listed in the U.S. Pharmacopeia or other generally recognized pharmacopeia for use in mammals, and more particularly in humans.

The term “about” or “approximately” usually means within 20%, alternatively within 10%, including within 5% of a given value or range. Alternatively, especially in biological systems, the term “about” means within about a log (i.e., an order of magnitude), including within a factor of two of a given value.

The compositions of the present invention may be used for the manufacture of a medicament for the treatment of at least one of the mentioned disorders, wherein the medicament is adapted to or appropriately prepared for a specific administration as disclosed herein (e.g., to once-a-day, twice-a-day administration, or three times a day administration). For this purpose the package leaflet and/or the patient information contains corresponding information.

### EXAMPLES

The following examples illustrate the invention without limiting its scope.

### Example 1

Complexes between neramexane mesylate and either hydroxypropyl-beta-cyclodextrin (HPBCD; grade: Kleptose™ HPB) or hydroxypropyl-gamma-cyclodextrin (HPGCD; grade: Cavasol™ W8 HP Pharma) are formed and investigated by isothermal titration calorimetry (ITC). The experiments are performed on a Microcal MCS-ITC instrument. The procedure consists of iteratively adding small portions of a test solution (here: a neramexane mesylate solution) through a motorised microlitre syringe to a receptor solution (here: a cyclodextrin solution) located in the calorimeter. Upon complexation, an exothermic or endothermic signal is produced which represents the sum of all thermal events including complexation itself, but also heats of dilution or (de)protonation. The titration curves are evaluated with the Microcal Origin software V2.9 with its original data evaluation module for titration calorimetric evaluations.

The experiments are carried out using aqueous phosphate buffer of pH 6 as a solvent. Solutions of neramexane mesylate, HPBCD, and HPGCD of various concentrations are prepared, and a number of titrations are conducted using different maximal molar ratios of neramexane to cyclodextrin (after complete addition of the drug substance), ranging from 1.25 to 5. Titrations with solvent solutions alone and in combination with cyclodextrin solution are performed to obtain curves indicating the heats of dilution, which may be used as baseline data for the correction of the complexation curves.

In all cases complexation occurs spontaneously. In the case of HPBCD, it is an exothermic event, whereas it is endothermic in the case of HPGCD. Moreover, the stoichiometry of binding as well as the calculated equilibrium constants are dependent on the final (maximal) molar ratio of neramexane to the cyclodextrin. In the case of HPBCD, for example, at a final molar ratio of 5, the stoichiometry of binding (of neramexane per cyclodextrin molecule) is 0.84 with an equilibrium constant of 788 M⁻¹, whereas at a final molar ratio of 1.25, the stoichiometry is 0.4 and the equilibrium constant 413 M⁻¹. An example of a result with HPGCD obtained with a final molar ratio of 1.8 results in a stoichiometry of 0.93 and an equilibrium constant of 112 M⁻¹.

### Example 2

Equimolar amounts of neramexane mesylate and either hydroxypropyl-beta-cyclodextrin (HPBCD; grade: Kleptose™ HPB), hydroxypropyl-gamma-cyclodextrin (HPGCD; grade: Cavasol™ W8 HP Pharma) or unsubstituted beta-cyclodextrin (BCD; grade: Kleptose™) are dissolved together in water and subsequently dried by vacuum evaporation in a Rotavapor apparatus. Samples of the resulting materials are analysed by solid state NMR (nuclear magnetic resonance) spectroscopy and some of them also by XRD (X-ray diffraction). In short, by NMR spectroscopy it may be shown that the samples indeed comprise true complexes of the drug substance with the cyclodextrins, and by XRD it is demonstrated that the complexes with HPBCD are amorphous, but those with BCD are crystalline.

The NMR experiments are conducted as one-dimensional and two-dimensional solid state NMR, wherein the two-dimensional technique is double-quantum/single-quantum correlation spectroscopy as described by S P Brown and H W Spiess (Advanced solid-state NMR methods for the elucidation of structure and dynamics of molecular, macromolecular, and supramolecular systems, Chemical Reviews 101, 4125-4155, 2001). In the series of experiments, the a.m. samples are compared to physical mixtures of neramexane mesylate and the respective cyclodextrin. Spectra are measured with two double-quantum excitation times (22 and 44 μs). In the case of the test samples (obtained by drying the neramexane/cyclodextrin solutions), clear cross-peaks are observed which indicate that at least some of the methyl protons of neramexane mesylate (1.1 ppm) must be very closely positioned with respect to some of the protons of the cyclodextrin (e.g. 4.2 ppm for BCD). In the case of the neramexane-BCD complex, these cross-peaks are at approx. 1 ppm in the single-quantum dimension and at approx. 5 ppm in the double-quantum dimension for both excitation times. In contrast, the corresponding spectra of the respective physical mixture have no evidence of any cross-peaks even at the longer excitation time. Hence, the cross-peaks of the complex indicates true complexation rather than being an artifact due to an overlap of broad peaks.

### Example 3

Aqueous liquid compositions comprising neramexane mesylate and hydroxypropyl-beta-cyclodextrin (HPBCD, grade: Kleptose™ HPB) are prepared by dissolving weighed amounts of the active compound and the cyclodextrin in measured amounts of sterile water. Subsequently, the remaining excipients are added under stirring. Sterile water is then added until the final volume is reached. The solutions are filled into 5 ml glass bottles. In addition, a solution without cyclodextrin is prepared for comparative purposes. Table 1 shows the compositions of test solutions; all amount in g unless stated otherwise.

Test solution A exhibits a molar cyclodextrin/neramexane ratio of 1:1, test solution B a ratio of 2:1.

TABLE 1

| Component | Solution A | Solution B | Solution C |
|---|---|---|---|
| Neramexane | 5.00 | 5.00 | 5.00 |
| mesylate | | | |
| HPBCD | 28.99 | 57.98 | — |
| Propylene glycol | 129.50 | 129.50 | 129.50 |
| Citrate buffer pH | 50.00 | 50.00 | 50.00 |
| 5.0 | | | |
| Peppermint flavour | 0.45 | 0.45 | 0.11 |
| Sterile water | ad 500 ml | ad 500 ml | ad 500 ml |

### Example 4

Samples of the test solutions prepared in Example 3 are blinded and tested for palatability by 18 persons. Rated on a scale of 1 to 6 (1 being the best grade), the average grade for the taste of solution A is 3.0, that of solution B is 2.7. The taste is described as barely bitter or not bitter at all. The smell is generally found to be almost neutral. In contrast, the average grade for solution C is 3.9 and is described as very bitter and stinging.

### Example 5

Samples of the test solutions prepared in Example 3 are evaluated in respect of their microbiological properties. For this purpose, the test of the European Pharmacopoeia IV, “Efficacy of antimicrobial preservation” (5.1.3.) is conducted. In this test, the samples are contaminated with substantial amounts of five major microbial contaminants (Escherichia coli, Pseudomonas aeruginosa, Staphylococcus aureus, Candida albicans, Aspergillus niger), incubated over 14 and 28 days, respectively, and then analysed for the same germs. In result, all of the test solutions fulfill the criteria for efficient preservation of oral formulations.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

1-14. (canceled)

15. A composition comprising a compound selected from those of formula (I) 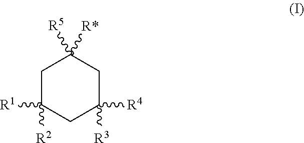
wherein R* is —(CH₂)ₙ—(CR⁶R⁷)ₘ—NR⁸R⁹
wherein n+m=0, 1, or 2
wherein R¹ through R⁷ are independently selected from the group consisting of hydrogen and C₁₋₆alkyl, wherein R⁸ and R⁹ are independently selected from the group consisting of hydrogen and C₁₋₆alkyl or together represent lower-alkylene —(CH₂)ₓ— wherein x is 2 to 5, inclusive, and optical isomers, enantiomers, hydrates, solvates, polymorphs, and pharmaceutically-acceptable salts thereof;
and a pharmaceutically acceptable cyclodextrin or combination of pharmaceutically acceptable cyclodextrins.

16. The composition according to claim 1, wherein the composition is an aqueous liquid composition.

17. The composition according to claim 1, wherein the composition is a semi-solid composition.

18. The composition according to claim 1, wherein the composition is a solid composition.

19. The composition according to any preceding claim, wherein the pharmaceutically acceptable cyclodextrin is selected from alpha-cyclodextrin, beta-cyclodextrin, randomly methylated beta-cyclodextrin, 2-O-methyl-beta-cyclodextrin, heptakis-(2,6-di-O-methyl)-beta-cyclodextrin (dimethyl-beta-cyclodextrin), acetylated dimethyl-beta-cyclodextrin, heptakis-(2,3,6-tri-O-methyl)-beta-cyclodextrin (trimethyl-beta-cyclodextrin, 2-hydroxypropyl-beta-cyclodextrin, sulfoalkylether-beta-cyclodextrin, sulfobutylether-beta-cyclodextrin, O-carboxymethyl-O-ethyl-beta-cyclodextrin, glucuronyl-glucosyl-beta-cyclodextrin, glucosyl-beta-cyclodextrin, maltosyl-beta-cyclodextrin, beta-cyclodextrin sulphate, beta-cyclodextrin phosphate, gamma-cyclodextrin, 2-hydroxypropyl-gamma-cyclodextrin, sulfoalkylether-beta-cyclodextrin, and sulfobutylether-beta-cyclodextrin.

20. The composition according to claims 15, wherein the pharmaceutically acceptable cyclodextrin is selected from optionally hydroxyalkyl-substituted beta- and gamma-cyclodextrins.

21. The composition according to claim 15, wherein the compound of formula (I) is neramexane or a pharmaceutically acceptable salt thereof.

22. The composition according to claim 15, wherein the molar ratio of the cyclodextrin to the compound of formula (I) is at least about 0.1:1.

23. The composition according to claim 15, wherein the concentration of the compound of formula (I) is in the range from about 2 mg/ml to about 100 mg/ml.

24. The composition according to claim 18, wherein the composition is an orally disintegrating dosage form or a formulation for reconstitution optionally in form of a powder, granules or a lyophilisate.

25. The composition according to claim 24, wherein reconstitution with an aqueous solvent results in an aqueous liquid composition.

26. A method of treating a CNS disorder or a condition selected from hypoxia, hypoglycemia, hepatic encephalopathy, chronic neurodegenerative diseases, dementia, Alzheimer's disease, vascular dementia, Parkinson's disease, Huntington's disease, multiple sclerosis, amyotrophic lateral sclerosis, AIDS-neurodegeneration, AIDS-related dementia, olivopontocerebellar atrophy, Tourette's syndrome, motor neurone disease, mitochondrial dysfunction, Korsakoff syndrome, Creutzfeldt-Jakob disease, chronic pain, acute pain, drug tolerance, dependence and addiction (e.g., opioids, cocaine, benzodiazepines, and alcohol), neuropathic pain, epilepsy, depression, anxiety, schizophrenia, spasticity, nystagmus, ocular diseases, tinnitus, hepatic encephalopathy, multiple sclerosis, stroke, dyskinesia, malaria, and viral infections such as hepatitis C and Borna virus, conditions requiring an immunomodulator, emesis, drug and alcohol abuse disorders, cognitive disorders, cerebellar tremor, and appetite disorders in a subject in need thereof, comprising administration of an effective amount of a composition according to claim 15.
